(19) United States
(12) Patent Application Publication
(10) Pub. No.: US 2016/0186916 A1
(43) Pub. Date: Jun. 30, 2016

(54) Algae-Based Method of Inhibiting Corrosion in Offshore Flexible Pipes

(71) Applicants: Venkat Ramanan Krishnan, Houston, TX (US); Stefanie Lynn Asher, Spring, TX (US)

(72) Inventors: Venkat Ramanan Krishnan, Houston, TX (US); Stefanie Lynn Asher, Spring, TX (US)

(21) Appl. No.: 14/937,268

(22) Filed: Nov. 10, 2015

Related U.S. Application Data
(60) Provisional application No. 62/097,916, filed on Dec. 30, 2014.

(51) Int. Cl. F16L 58/18; A01G 33/00; C12N 1/12

(52) U.S. Cl. CPC F16L 58/18; C12N 1/12; A01G 33/00

(57) ABSTRACT

A method of inhibiting the corrosion of a flexible pipe having a breach, the flexible pipe having an inner and an outer polymer sheath forming an annulus, the annulus having a plurality of metal armor wires, the method comprising injecting an algae strain into the annulus, wherein the algae strain is effective to adjust at least one of the oxygen level, CO₂ level, H₂S level, and pH level within the annulus and inhibit the corrosion of the armor wires. An anticorrosion kit for inhibiting the corrosion of a flexible pipe having a breach is also provided.

### CROSS-REFERENCE TO RELATED APPLICATION

This application claims the priority benefit of U.S. Provisional Patent Application 62/097,916, filed Dec. 30, 2014, entitled ALGAE-BASED METHOD OF INHIBITING CORROSION IN OFFSHORE FLEXIBLE PIPES, the entirety of which is incorporated by reference herein.

### FIELD

The present disclosure relates to a method of inhibiting corrosion and, more particularly, to the inhibition of corrosion in the internal metal components of flexible pipes used in the loading and unloading of produced fluids, liquid products, or the like, to and from sea-going vessels.

### BACKGROUND

Offshore floating production systems often employ flexible risers for producing reservoir well fluids or injecting water or carbon dioxide (CO₂) into the reservoir. The flexible risers are especially useful over steel risers in offshore environments with dynamic loading environments due to winds, waves, and currents.

The structure of flexible pipes typically includes inner and outer polymer sheaths and steel tensile and pressure armor wires to carry the structural loads. The space between the inner and outer polymer sheaths of such flexible pipes is called the annulus, which houses steel armor wires having gaps between them. These air gaps can equal up to about 8% of the annulus volume. One of the challenges with using these flexible pipes is the prevention of the corrosion of the metal armor wires within the pipe annulus. Corrosion could result or expedite armor wire failure, by fracture or otherwise, and potentially result in structural failure of the flexible riser itself.

External sheath damage in a flexible pipe riser occurs most commonly in the splash zone within an I-tube, a J-tube, or similar hang-off structure near the platform or offshore vessel structure. These locations are where the flexible pipe is likely to sustain damage to the outer sheath under the influence of wave loading, due to dynamic contact against the tubes to which it is adjacent. Damage to the external sheath can also occur during manufacturing or installation of the flexible pipes, when the softer outer sheath may get caught and ripped by sharp objects, such as nails, left lying around. During operations, the outer sheath is likely to get damaged by dropped objects or from banging against other risers within the moon pool. Damage to the outer sheath in the splash zone can result in the corrosive sea water entering the annulus, and, coupled with free oxygen, this can cause corrosion of the armor wires.

Diffusion of production fluid and/or gas from the bore can also occur during operations, which upon condensation can result in a corrosive environment within the annulus. The internal sheath is typically made of a polymeric material that cannot prevent this diffusion. If the produced fluid consists of sour or sweet gas, this can also result in the corrosion of the armor wires.

Once the annulus is subjected to corrosion, it is difficult to protect the exposed metallic components. Corrosion of these metallic layers affects operations integrity, may result in a halt to production, and/or shortens the lifetime of flexible pipes deployed offshore. Cathodic protection has, at best, a limited and typically no effect in the splash zone, due to the intermittent exposure to water. Traditional corrosion inhibiting is challenging due to difficulties in supplying constant chemical inhibitor to the exposed metal within the annulus. Repair methodologies to recoat the damaged sheath are available but typically trap some of the corrosive environment internally, thereby creating a localized corrosion environment.

As such, there is a need for a cost-effective solution to the corrosion problem encountered with the flexible pipes used in the loading and unloading of produced fluids, liquid products, or the like, to and from sea-going vessels.

### SUMMARY

In one aspect, disclosed herein is a method of inhibiting the corrosion of a flexible pipe having a breach, the flexible pipe having at least an inner and an outer polymer sheath forming an annulus, the annulus including a plurality of metal armor wires. The method includes injecting an algae strain into the annulus, wherein the algae strain is effective to adjust at least one of the oxygen level, CO₂ level, hydrogen sulfide (H₂S) level, and pH level within the annulus and inhibit the corrosion of the armor wires.

In some embodiments, the method further comprises identifying the location of the breach in the flexible pipe and selecting the algae strain on the basis of environmental conditions within the flexible pipe at the location.

In some embodiments, the breach may be an external breach and the algae strain may be injected into the annulus through the external breach.

In some embodiments, the algae strain may be injected into the annulus by forming an injection port in the outer polymer sheath of the flexible pipe proximate the breach and injecting the algae strain into the annulus via the injection port. The breach may be an internal breach and the injection port may be located proximate the internal breach.

In some embodiments, the method further comprises forming a live-algae passivation layer on the surfaces of the armor wires reducing the free surface area of the armor wires.

In some embodiments, the method further comprises genetically modifying algae to produce a corrosion-inhibiting algae strain.

In some embodiments, the method further comprises providing a supplement of simple carbohydrates or organic carbon within the annulus to sustain the algae strain.

In yet another aspect, disclosed herein is an anticorrosion kit for inhibiting the corrosion of a flexible pipe having a breach, the flexible pipe having an inner and outer polymer sheath forming an annulus, the annulus having a plurality of metal armor wires. The kit includes at least one algae strain selected on the basis of environmental conditions within the flexible pipe, wherein the algae strain is effective to adjust at least one of the oxygen level, CO₂ level, H₂S level and pH level within the annulus and inhibit the corrosion of the armor wires.

In some embodiments, the kit further comprises a supplement of simple carbohydrates or organic carbon to sustain the algae strain.

In some embodiments, the algae strain forms a passivation layer that reduces the free surface area of the armor wires.

In some embodiments, an environmental condition is the lack of available sunlight.

In some embodiments, the algae strain selected comprises a heterotrophic algae strain.

In some embodiments, the algae strain selected comprises Spirulina platensis.

In some embodiments, the algae strain selected comprises a mixotropic algae strain.

In some embodiments, the metal armor wire is comprised of steel.

In some embodiments, an environmental condition is the presence of available sunlight.

In some embodiments, the algae strain selected comprises aerobic algae.

In some embodiments, the algae strain produces a quatamine corrosion inhibitor byproduct.

In some embodiments, the algae strain selected utilizes organic carbon and CO₂ during photosynthesis and respiration.

### BRIEF DESCRIPTION OF THE DRAWINGS

FIG. 1 presents a cutaway illustration of one example of a flexible pipe, showing the layers of the flexible pipe.

FIG. 2 presents a flow chart of a method of inhibiting the corrosion of a flexible pipe having an external or internal breach, the flexible pipe having an inner and outer polymer sheath forming an annulus, the annulus having a plurality of metal armor wires, in accordance with the present disclosure.

### DETAILED DESCRIPTION

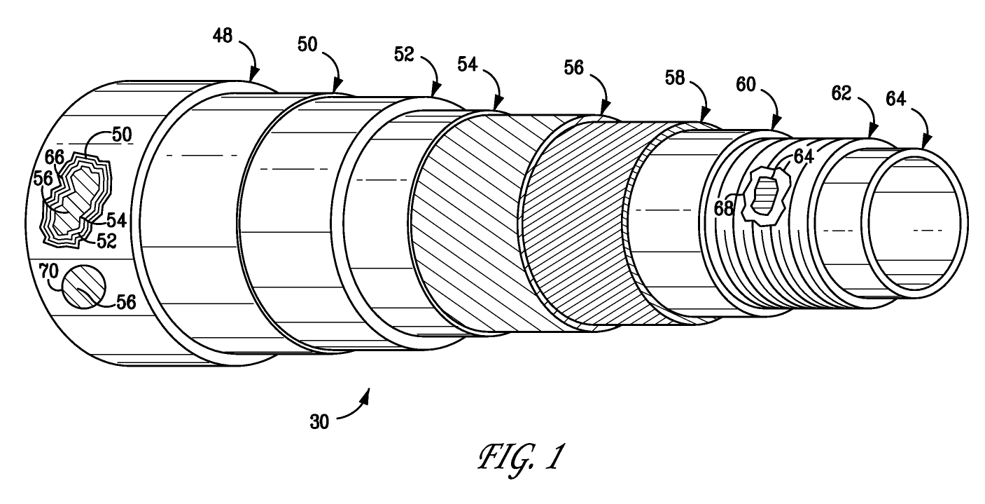

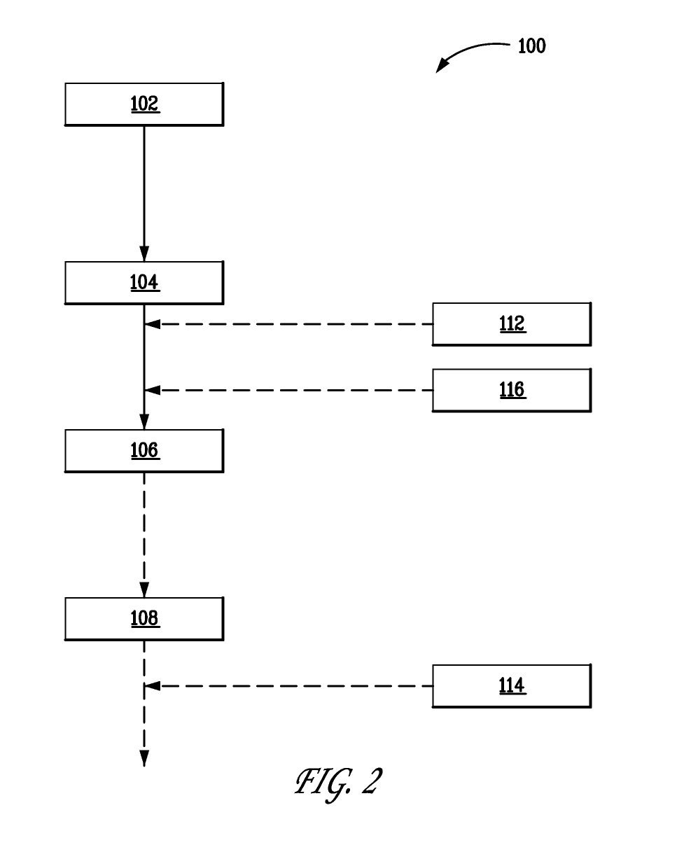

FIGS. 1 and 2 provide illustrative, non-exclusive examples relating to a method and kit for inhibiting the corrosion of a flexible pipe having a breach, the flexible pipe having an inner and outer polymer sheath forming an annulus, the annulus having a plurality of metal armor wires, according to the present disclosure, together with elements that may include, be associated with, be operatively attached to, and/or utilize such a method or kit.

In FIGS. 1 and 2, like numerals denote like, or similar, structures and/or features; and each of the illustrated structures and/or features may not be discussed in detail herein with reference to the figures. Similarly, each structure and/or feature may not be explicitly labeled in the figures; and any structure and/or feature that is discussed herein with reference to the figures may be utilized with any other structure and/or feature without departing from the scope of the present disclosure.

In general, structures and/or features that are, or are likely to be, included in a given embodiment are indicated in solid lines in the figures, while optional structures and/or features are indicated in broken lines. However, a given embodiment is not required to include all structures and/or features that are illustrated in solid lines therein, and any suitable number of such structures and/or features may be omitted from a given embodiment without departing from the scope of the present disclosure.

Although the approach disclosed herein can be applied to a variety of undersea fluid handling system designs and operations, the present description will primarily be related to inhibiting the corrosion of a flexible pipe having a breach, the flexible pipe having an inner and outer polymer sheath forming an annulus, the annulus having a plurality of metal armor wires.

By “corrosion” is meant the reaction of an engineering metal with its environment with a consequent deterioration in properties of the metal. In other words, corrosion can be defined as the chemical reaction between a metal surface and its environment.

By “algae” is meant an aquatic, eucaryotic single cell or multicellular plant without stems, roots, and leaves that is typically autotrophic, which grows in bodies of water, including fresh water, sea water, and brackish water, with the degree of growth being in relative proportion to the amount of nutrients available.

As may be appreciated, the present invention will benefit a wide variety of underwater fluid handling systems, such as a flexible riser system, employing one or more flexible pipes susceptible to forming breaches over time, wherein the flexible pipe has an inner and outer polymer sheath forming an annulus, the annulus enclosing a plurality of metal armor wires susceptible to corrosion upon encountering such a breach.

Referring now to FIG. 1, a cutaway illustration of one version of a flexible pipe 30 is presented. Typically, flexible pipe 30 has an outside diameter as small as possible to limit its resistance to currents.

One supplier of flexible pipe 30 suitable for use in offshore exploration and production is Technip of Paris, France. As those skilled in the art will recognize, commercially available flexible pipe is often designed to meet API-17J, a Specification for Unbonded Flexible Pipe.

As indicated, the flexible pipe 30 will have at least an inner and outer polymer sheath forming an annulus, the annulus enclosing a plurality of metal armor wires. Typically, the metal armor wires comprise steel. In some embodiments, the metal armor wires are comprised of high strength carbon steel.

Commercially available flexible pipe for undersea applications comes in a variety of configurations, several of which include multiple protective layers. In one form, flexible pipe 30 may have as many as nine layers, as shown in FIG. 1. An outer layer 48 of flexible pipe 30 may be a thermoplastic elastomer and serve as a protective sheath. The second layer 50 may be selected from a variety of materials and, in one form, may be a Kevlar® fabric tape. The third layer 52 may be formed from a polyolefin, such as polyethylene, and serve as a sheath. Inside the polyolefin sheath 52 may be another layer of Kevlar® fabric tape, serving as the fourth layer 54.

In the embodiment depicted in FIG. 1, the fifth layer 56 is an outside metal armor layer and the sixth layer 58 is an inside metal armor layer. Optionally, a seventh layer 60 may be provided. The seventh layer 60 may be formed of another steel wire product. An eighth layer 62 may be provided to serve as a pressure sheath. Eighth layer 62 may comprise a high performance polyamide, such as Rislan® polyamide. Finally, a ninth layer 64 may be provided, the ninth layer 64 forming a stainless steel inside layer.

As may be appreciated, the flexible pipe 30 of FIG. 1 has, in addition to the conventional polyolefin (polyethylene) external sheath of third layer 52, a reinforced protective sheath made of thermoplastic elastomer (first layer 48). Both sheaths are separated by layers of Kevlar® fabric tape (50 and 54). The thermoplastic elastomer protective sheath 48 serves to resist abrasion and the Kevlar® fabric tape layers (50 and 54) mechanically reinforce the external sheath and protect it from damage.

Despite efforts to improve flexible pipe design, the prevention of corrosion of the metal armor wires within the pipe annulus remains an issue. As mentioned above, corrosion may result in armor wire failure, by fracture or otherwise.

External sheath damage in a flexible pipe riser occurs most commonly in the splash zone within an I-tube, a J-tube, or similar hang-off structure near the platform or offshore vessel structure. These locations are where the flexible pipe is likely to sustain damage to the outer sheath under the influence of wave loading, due to dynamic contact against the tubes to which it is adjacent. Damage to the external sheath can also occur during manufacturing or installation of the flexible pipes, when the softer outer sheath may get caught and ripped by sharp objects, such as nails, left lying around. During operations, the outer sheath is likely to get damaged by dropped objects or from banging against other risers within a moon pool. Damage to the outer sheath in the splash zone can result in the corrosive salt water entering the annulus, and, coupled with free oxygen, this can cause corrosion of the armor wires.

Diffusion of production fluid and/or gas from the bore can also occur during operations, which upon condensation can result in a corrosive environment within the annulus. If the produced fluid consists of sour or sweet gas, this can also result in corrosion of the armor wires. As may be appreciated by those skilled in the art, once the annulus is subjected to corrosion, it is difficult to protect the exposed metallic components. Corrosion of these metallic layers affects operations integrity, may result in a halt to production, and/or shortens the lifetime of flexible pipes deployed offshore.

To address the aforementioned issues, disclosed herein is a method of inhibiting the corrosion of a flexible pipe 30 having an external breach 66, the flexible pipe 30 having at least an inner polymer sheath 62 and outer polymer sheath 52 forming an annulus, the annulus including a plurality of metal armor wires 56 and 58. The location of an external breach 66 or an internal breach 68 in the flexible pipe 30 may be identified and an algae strain selected on the basis of environmental conditions within the flexible pipe 30 at the location. The algae strain is injected into the annulus, the algae strain effective to adjust at least one of the oxygen level, CO₂ level, H₂S level, and pH level within the annulus and inhibit the corrosion of the armor wires 56 and 58.

The algae lifecycle can be inhibitive to the presence of oxygen, since algae use up oxygen to make their food during the process of photosynthesis. However, in deep waters, the sunlight penetrated sea does not extend down to the level of the seabed. In applications where there is a lack of sunlight, algae may be genetically modified in order to produce heterotrophic algae that do not require sunlight and can subsist on simple carbohydrates. The use of algae can help inhibit the corrosion through slowdown of the chemical degradation mechanisms associated with corrosion. Genetically modified algae or selective species tuning can be accomplished to supply an algae that produces a corrosion inhibitor by-product, such as a quatamine. The addition of an algae-based inhibitor provides further benefits by resulting in the formation of a slimy (live algae) passivation layer that can, in turn, inhibit corrosion by reducing the free surface area of the wires.

The algae can be introduced through the damaged portion of the outer sheath by means of divers in the splash zone region or remotely operated underwater vehicles (ROVs) in deeper locations.

Still referring to FIG. 1, to address cases where there has been an internal breach 68 of the inner sheath 62, an injection port 70 may be formed in the outermost polymer sheath 52 of the flexible pipe 30 to access the annulus. The algae strain may be injected into the injection port 70, the algae strain effective to adjust at least one of the oxygen level, CO₂ level, H₂S level and pH level within the annulus and inhibit the corrosion of the armor wires 56 and 58. As may be appreciated, the method disclosed can be effective for cases where corrosion is detected in the annulus because of gas seepage through the internal sheath 62.

For the case where the breach is within the zone receiving sunlight, the algae employed will sustain itself by feeding on oxygen and sunlight in the splash zone region. For the case where the breach is below the zone receiving sunlight, heterotrophic algae can be sustained through supplements of simple carbohydrates. When the algae starts to die off, due to lack of sunlight, an extreme cold front, storms, etc., it leaves behind a large amount of dead organic matter. This organic matter may be decomposed by microorganisms. With the added organic matter load, the total amount of decomposition occurring increases and the decomposition process uses up oxygen and gives off CO₂. Strain selection of algae should therefore be considered in order to limit the introduction of CO₂ in a water system.

The use of an algae-based inhibitor can provide a lower cost substitute to conventional water based corrosion inhibitors that require continual pumping through the annulus. The algae introduction can be a onetime event, since it is a self-sustaining life-form. As such, no additional maintenance costs would be required, other than regular ROV/diver inspections to ensure the presence of the algae and/or possible supplements or replenishments.

In some embodiments, the algae strain selected comprises Spirulina platensis. Spirulina platensis may be obtained in powdered form from a wide variety of sources. The powdered form may be extracted with ethanol using a Soxhlet apparatus. The ethanolic extract may then be distilled to obtain a solid residue, which may then be used as the inhibitor. The inhibitor may then be dissolved in a suitable medium, as those skilled in the art would plainly recognize.

In some embodiments, the algae strain forms a passivation layer that reduces the free surface area of the armor wires.

In some embodiments, the algae strain selected comprises a heterotrophic algae strain. In some embodiments, a supplement of simple carbohydrates or organic carbon is provided to sustain the algae strain.

In some embodiments, the algae strain selected comprises a mixotropic algae strain. In some embodiments, the algae strain selected comprises aerobic algae.

In some embodiments, the algae strain produces a quatamine corrosion inhibitor byproduct.

Referring now to FIG. 2, a flow chart of a method of inhibiting the corrosion of a flexible pipe having an external or internal breach is presented. As shown in FIG. 1, the flexible pipe 30 has an inner polymer sheath 62 and an outer polymer sheath 52 forming an annulus, the annulus having a plurality of metal armor wires 56 and 58. The method 100 includes 102, identifying the location of an external or internal breach in the flexible pipe; 104, selecting an algae strain on the basis of environmental conditions within the flexible pipe at the location; and 106, injecting the algae strain into the annulus, wherein the algae strain is effective to adjust at least one or the oxygen level, CO₂ level, H₂S level, and pH level within the annulus and inhibit the corrosion of the armor wires.

In some embodiments, the method 100 further includes 108, forming a live-algae passivation layer on the surfaces of the armor wires reducing the free surface area of the armor wires.

In some embodiments, the method 100 further includes 112, genetically modifying algae to produce a corrosion inhibiting algae strain. In some embodiments, the method 100 further includes 114, providing a supplement of simple carbohydrates or organic carbon to sustain the algae strain.

In some embodiments, the breach is an internal breach and the method 100 further includes 116, forming an injection port in the outer polymer sheath of the flexible pipe; and 106, injecting the algae strain into the annulus via the injection port.

Advantageously, an anticorrosion kit may be provided for field use to locations where flexible pipes of the types described may be employed. Such a field kit includes at least one algae strain selected on the basis of environmental conditions within the flexible pipe, wherein the algae strain is effective to adjust at least one of the oxygen level, CO₂ level, H₂S level, and pH level within the annulus and inhibit the corrosion of the armor wires. The algae of the field kit may be provided in powder form.

The embodiments disclosed herein, as illustratively described and exemplified hereinabove, have several beneficial and advantageous aspects, characteristics, and features. The embodiments disclosed herein successfully address and overcome shortcomings and limitations, and widen the scope, of currently known teachings with respect to flexible pipe corrosion inhibition.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the phrase “at least one,” in reference to a list of one or more entities, should be understood to mean at least one entity selected from any one or more of the entities in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently, “at least one of A and/or B”) may refer, in one embodiment, to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C” and “A, B, and/or C” may mean A alone, B alone, C alone, A and B together, A and C together, B and C together, A, B, and C together, and optionally any of the above in combination with at least one other entity.

In the event that any patents, patent applications, or other references are incorporated by reference herein and define a term in a manner or are otherwise inconsistent with either the non-incorporated portion of the present disclosure or with any of the other incorporated references, the non-incorporated portion of the present disclosure shall control, and the term or incorporated disclosure therein shall control only with respect to the reference in which the term is defined and/or the incorporated disclosure was originally present. Illustrative, non-exclusive examples of apparatus and methods according to the present disclosure have been presented.

### INDUSTRIAL APPLICABILITY

The apparatus and methods disclosed herein are applicable to the oil and gas industry.

The specific embodiments as disclosed and illustrated herein are not to be considered in a limiting sense, as numerous variations are possible. The subject matter of the inventions described herein includes all novel and non-obvious combinations and subcombinations of the various elements, features, functions, and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements, and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

What is claimed is:

1. A method of inhibiting the corrosion of a flexible pipe having a breach, the flexible pipe having an inner and an outer polymer sheath forming an annulus, the annulus having a plurality of metal armor wires, the method comprising:
injecting an algae strain into the annulus, wherein the algae strain is effective to adjust at least one of the oxygen level, CO₂ level, H₂S level, and pH level within the annulus and inhibit the corrosion of the armor wires.

2. The method of claim 1, further comprising identifying the location of the breach in the flexible pipe and selecting the algae strain on the basis of environmental conditions within the flexible pipe at the location.

3. The method of claim 1, wherein the breach is an external breach.

4. The method of claim 3, wherein the algae strain is injected into the annulus through the external breach.

5. The method of claim 1, further comprising forming an injection port in the outer polymer sheath of the flexible pipe proximate the breach and injecting the algae strain into the annulus via the injection port.

6. The method of claim 5, wherein the breach is an internal breach.

7. The method of claim 1, further comprising forming a live-algae passivation layer on the surfaces of the armor wires reducing the free surface area of the armor wires.

8. The method of claim 1, further comprising genetically modifying algae to produce a corrosion inhibiting algae strain.

9. The method of claim 1, wherein an environmental condition is the lack of available sunlight.

10. The method of claim 9, wherein the algae strain selected comprises a heterotrophic algae strain.

11. The method of claim 9, wherein the algae strain selected comprises Spirulina platensis.

12. The method of claim 9, wherein the algae strain selected comprises a mixotropic algae strain.

13. The method of claim 9, further comprising providing a supplement of simple carbohydrates or organic carbon within the annulus to sustain the algae strain.

14. The method of claim 1, wherein an environmental condition is the presence of available sunlight.

15. The method of claim 14, wherein the algae strain selected comprises aerobic algae.

16. The method of claim 1, wherein the algae strain produces a quatamine corrosion inhibitor byproduct.

17. The method of claim 1, wherein the algae strain selected utilizes organic carbon and CO₂ during photosynthesis and respiration.

18. The method of claim 1, wherein the metal armor wire is comprised of steel.

19. An anticorrosion kit for inhibiting the corrosion of a flexible pipe having a breach, the flexible pipe having an inner and an outer polymer sheath forming an annulus, the annulus having a plurality of metal armor wires, comprising at least one algae strain selected on the basis of environmental conditions within the flexible pipe, wherein the algae strain is effective to adjust at least one of the oxygen level, CO₂ level, H₂S level, and pH level within the annulus and inhibit the corrosion of the armor wires.
